(19) United States
(12) Patent Application Publication
(10) Pub. No.: US 2021/0223239 A1
(43) Pub. Date: Jul. 22, 2021

(54) Saliva Test Apparatus

(71) Applicants: Jordan Jacek de Haan, Denver, CO (US); William Jackson Bowen, III, Denver, CO (US); William George Norman Coon, Longmont, CO (US); Shane Kawika Vogt, Lafayette, CO (US)

(72) Inventors: Jordan Jacek de Haan, Denver, CO (US); William Jackson Bowen, III, Denver, CO (US); William George Norman Coon, Longmont, CO (US); Shane Kawika Vogt, Lafayette, CO (US)

(21) Appl. No.: 17/149,157

(22) Filed: Jan. 14, 2021

Related U.S. Application Data
(60) Provisional application No. 62/962,885, filed on Jan. 17, 2020.
(60) Provisional application No. 63/092,441, filed on Oct. 15, 2020.

(51) Int. Cl. G01N 33/543; B01L 3/00; G16H 10/40; G16H 10/60

(52) U.S. Cl. CPC G01N 33/54366; B01L 3/502; B01L 2400/0406; G16H 10/60; B01L 2300/0825; G16H 10/40

(57) ABSTRACT

An ancillary saliva testing apparatus for the presence of a selected substance utilizing an internet connected electronic device with a camera for third party review, the electronic device also having electronic communication to the apparatus, the apparatus has an enclosure that accommodates an insertable saliva test element assembly that includes a test element casing that secures a saliva test strip within the casing. The enclosure includes color sensing circuitry for the saliva test strip and communication circuitry from the color sensing circuitry to the electronic device. Also, an user interface application in on the electronic device to document a user inserting the casing into the enclosure with the camera verifying the user identity via facial recognition, also the saliva test strip that includes both an identification code and change of state test result color code on the saliva test strip based on the user wetting the test strip with saliva.

### RELATED PATENT APPLICATIONS

This patent application claims the benefit of U.S. provisional patent application Ser. No. 62/962,885 filed on Jan. 17, 2020 by Jordan Jacek De Haan of Denver, Colo., U.S. and William Jackson Bowen III of Boulder Colo., U.S. and this patent application also claims the benefit of U.S. provisional patent application Ser. No. 63/092,441 filed on Oct. 15, 2020 by Jordan Jacek de Haan of Denver, Colo., U.S., William Jackson Bowen III of Denver, Colo., U.S., William George Norman Coon of Longmont, Colo., U.S., and Shane Kawika Vogt of Lafayette, Colo., U.S.

### TECHNICAL FIELD

The overall field of this invention generally pertains to the field of biomolecular diagnostics, in particular to a portable point-of-collection diagnostic system, method, and components enabled to connect with a smart device accessory for tracking results and remotely sending data to an authorized third party.

### BACKGROUND OF INVENTION

Bodily fluids are frequently collected for analysis, the purpose of which can range from diagnosing a medical condition to checking for illicit or controlled drugs in an individual's system. Generally, many types of analysis may employ a test strip onto which a bodily fluid will be applied. The test strip will have reagents that will bind to or react with certain substances in the bodily fluid. In some cases, the test result may be determined by visually interpreting the test strip. In other cases, the test strip will be introduced into a device to aid with interpreting the result. However, there are generally no ways to ensure that the individual being tested has not tampered with the test strip or the results unless the test is conducted by another individual such as a nurse, technician, or at a police station among others. Further, these tests and devices generally do not allow the user to perform the test on their own and for them to subsequently remotely send it to the interested party who is monitoring the person under their supervision.

Thus, there remains a need for a device that is easy to use, is non-invasive, tamper-proof, and allows monitoring of an individual remotely while providing real time, documented test results to the requesting party. It is the object of the present invention to provide a device that allows monitoring a person under supervision without requiring in-person appointments. It is also the object of the present invention to provide a device that makes the person taking the test and being monitored to be accountable for themselves and also removes the person's ability to find a way to cheat their drug test. It is also the object to provide a test that is less invasive than collecting urine. It is also the object to provide real-time test results to a person that is requesting the information remotely.

The present disclosure recognizes the unsolved need for a low cost, easy to use, non-invasive, tamper-proof, remote supervised drug testing, ease of sharing results remotely, and a more efficient way to digitize file, and store drug test results. The saliva based drug testing device, as disclosed in one or more non-limiting embodiments, can have usages in many areas such as and not limited to sobriety programs, government, consumers, schools, and companies. In respect to sobriety programs, a drug and alcohol treatment program may use this device to monitor a client's abstinence from drugs and alcohol. The test results can be automatically compiled in one place. It is especially beneficial in monitoring clients who are allowed to leave the premises, to ensure their sobriety continues while being supervised remotely.

In government settings, the device can be used by probation and parole offices to monitor persons under their supervision, and do so remotely. The benefit also extends to parents or family members who may want to monitor their children or loved ones to ensure they are not using drugs. This allows family members to institute interventional measures, such as treatment or other suitable course of action. Schools and companies can also monitor their students and employees. For example, companies may need to monitor their employees when they travel for work, such as truck drivers to ensure proper protocol is followed and safe drivers are on the road. In all these situations, the beneficial features are that monitoring can be done anywhere and does not require the individual to be tested to present themselves to an individual to be tested. The apparatus is also beneficial because it ensures real time drug testing results are available to the monitoring party remotely, and it also removes the ability of the person being tested to find a way to cheat their drug test.

### SUMMARY OF INVENTION

Broadly, the present invention is an ancillary saliva testing apparatus for helping to indicate the presence of a selected substance that utilizes an internet connected electronic device with a camera, wherein the electronic device also having electronic communication to the saliva testing apparatus, the saliva testing apparatus includes a saliva test element assembly that includes a test element casing and a consumable saliva test strip that is disposed within the test element casing.

Further included in the ancillary saliva testing apparatus is an enclosure that includes a receiving port for said test element casing, the enclosure further includes color sensing circuitry for the consumable saliva test strip, and communication circuitry from the color sensing circuitry to the electronic device.

In addition for the ancillary saliva testing apparatus a user interface application is included on the electronic device for the enclosure to operationally facilitate dynamic and continuous documenting of a user using facial recognition, plus documentation of the user saliva wetting into the test element casing with the electronic device camera verifying the user identity through the facial recognition. Also for the saliva test strip that includes both an identification code on the saliva test strip and change of state test result color code on the saliva test strip after the user wetting with saliva, wherein the color sensing circuitry communicates the wetted saliva test strip image to the user interface application through the communication circuitry displaying saliva strip test results from selected saliva test strip test result criteria. Wherein the test results are further communicated to an internet portal for a third party to have access to the saliva testing results.

These and other objects of the present invention will become more readily appreciated and understood from a consideration of the following detailed description of the exemplary embodiments of the present invention when taken together with the accompanying drawings, in which;

### BRIEF DESCRIPTION OF DRAWINGS

FIG. 1 shows an upper perspective view of the test stand that is in the folded state;

FIG. 2 shows an upper perspective view of the test stand that is in the opened state;

FIG. 3 shows a side elevation view of the frustoconical channel for directing saliva into the cartridge, also shown is the saliva test strip disposed within the cartridge, further the saliva test strip identification code;

FIG. 4 shows an upper perspective view of the frustoconical channel for directing saliva into the cartridge, also shown is one of the saliva test strips raised for pictorial clarity, with the saliva test strips all disposed within the cartridge, further the saliva test strip identification code is shown and the lower portion of the test strip displaying the color coded test results from the saliva reacting with the test strip;

FIG. 5 shows an upper perspective view of the test stand in the opened state wherein an electronic device is disposed in the left side of the stand with the electronic device including a camera facing to the right, further the frustoconical channel for directing saliva into the cartridge is shown disposed in the right side of the test stand wherein the camera faces the cartridge to record the user through facial recognition and as shown to record the image of the saliva test strip identification code and the saliva test strip color coded test results;

FIG. 6 shows an upper perspective view of the test stand in the opened state wherein the electronic device is disposed in the left side of the stand with the electronic device including the camera facing to the right, further the frustoconical channel for directing saliva into the cartridge is shown disposed in the right side of the test stand wherein the camera faces the cartridge to record the user through facial recognition and as shown to record the image of the saliva test strip identification code and the saliva test strip color coded test results, further the display screen of the electronic device a menu for test instructions as part of the user interface;

FIG. 7 being a continuation of FIG. 6 shows an upper perspective view of the test stand in the opened state wherein the electronic device is disposed in the left side of the stand with the electronic device including the camera facing to the right, further the frustoconical channel for directing saliva into the cartridge is shown disposed in the right side of the test stand wherein the camera faces the cartridge to record the user through facial recognition and as shown to record the image of the saliva test strip identification code and the saliva test strip color coded test results, further the display screen of the electronic device a menu for test instructions as part of the user interface with the actual saliva test sample shown going into the frustoconical channel;

FIG. 8 being a continuation of FIG. 6 shows a reverse upper perspective view of the test stand in the opened state wherein the electronic device is disposed in the left side of the stand with the electronic device including the camera facing to the right, further the frustoconical channel for directing saliva into the cartridge is shown disposed in the right side of the test stand wherein the camera faces the cartridge to record the user through facial recognition and as shown to record the image of the saliva test strip identification code and the saliva test strip color coded test results, with the actual saliva test sample shown going into the frustoconical channel;

FIG. 9 shows an upper perspective view of the test stand in the opened state wherein the electronic device is disposed in the left side of the stand with the electronic device including the camera facing to the right, further the frustoconical channel for directing saliva into the cartridge is shown disposed in the right side of the test stand wherein the camera faces the cartridge to record the user through facial recognition and image of the saliva test strip identification code and the saliva test strip color coded test results, further the display screen of the electronic device a menu for test instructions as part of the user interface, also shown in the electronic device utilizing cloud storage for test results and review by third parties;

FIG. 10 shows an upper perspective view of the ancillary saliva testing apparatus that includes the saliva test element assembly, color sensing circuitry, communication circuitry all disposed within an enclosure, also shown is the electronic device that provides a user interface with the menu shown on the display screen, wherein the electronic device communicates with the ancillary saliva testing apparatus and cloud storage for test results and review by third parties, further the electronic device includes a camera that uses facial recognition to identify the user of the ancillary saliva testing apparatus;

FIG. 11 shows a perspective view of the enclosure interior that includes the color sensing circuitry, and a portion of the receiving port of the enclosure for the saliva test element assembly;

FIG. 12 shows a perspective view of the enclosure interior that includes the communication circuitry, and a portion of the receiving port of the enclosure for the saliva test element assembly;

FIG. 13 shows a perspective and partial cross sectional view of the saliva test element assembly that includes the element casing, the saliva cavity disposed within the element casing, the saliva test strip disposed within the element casing, and the saliva test strip identification, plus indication of a selected substance on the saliva test strip;

FIG. 14 shows a perspective view of the self-contained testing apparatus that includes the camera body that uses facial recognition for user identification, also included is the perceptible output, the body control circuitry, and the internet communication circuitry, all disposed within the body;

FIG. 15 shows a top view of the self-contained testing apparatus that includes the camera body, the port for the saliva test cartridge, all disposed within the body;

FIG. 16 shows a side elevation view of the frustoconical channel for directing saliva into the cartridge, also shown is the saliva test strip disposed within the cartridge, further the saliva test strip identification code;

FIG. 17 shows a perspective view of the structural integrity indicator for the cartridge or casing;

FIG. 18 shows a perspective view of the self-contained testing apparatus that includes the camera body disposed in the body, with the frustoconical channel and test strip cartridge inserted into the body port;

FIG. 19 shows a perspective use view of the self-contained testing apparatus that includes the camera body disposed in the body that uses facial recognition for user identification, the perceptible output, with the frustoconical channel and test strip cartridge inserted into the body port, also shown is the cloud storage for test results and review by third parties;

FIG. 20 shows a top view of the frustoconical channel for directing saliva into the cartridge, also shown is the saliva cavity for the cartridge, wherein the saliva cavity is disposed within the cartridge, further shown is the saliva is directed into the saliva cavity;

FIG. 21 shows a perspective cross section 21-21 from FIG. 20, wherein FIG. 21 shows the frustoconical channel for directing saliva into the cartridge, also shown is the saliva cavity for the cartridge, wherein the saliva cavity is disposed within the cartridge, also shown is the saliva test strip disposed within the cartridge, further the saliva test strip identification code is shown with the lower portion of the test strip displaying the color coded test results for presence of the selected substance from the saliva reacting with the test strip;

FIG. 22 shows a cross section 22-22 from FIG. 20, wherein FIG. 22 shows the frustoconical channel for directing saliva into the cartridge, also shown is the saliva cavity for the cartridge, wherein the saliva cavity is disposed within the cartridge, also shown is the saliva test strip disposed within the cartridge, further the saliva test strip identification code, with the lower portion of the test strip displaying the color coded test results for presence of the selected substance from the saliva reacting with the test strip; and

FIG. 23 shows a another perspective cross section 23-23 from FIG. 20, wherein FIG. 23 shows the frustoconical channel for directing saliva into the cartridge, also shown is the saliva cavity for the cartridge, wherein the saliva cavity is disposed within the cartridge, also shown is the capability of the cartridge holding a plurality of saliva test strips that are disposed within the cartridge, further the saliva test strip identification code(s), with the lower portion of the test strip displaying the color coded test results for presence of the selected substance(s) from the saliva reacting with the test strip(s).

### REFERENCE NUMBERS IN DRAWINGS

50 Ancillary saliva testing apparatus
55 Presence of a selected substance(s) on the test strip 120 via color coding from saliva 125 reacting on the test strip 120, also a QR code could be present in this same area for test strip 120 identification
60 Electronic device
65 Internet connection of the electronic device 60
70 Camera of the electronic device 60
75 Electronic communication of the electronic device 60
80 Internet portal for review by a third party
85 Saliva test element assembly
90 Saliva test element casing for securing the saliva test strip 120 that can be used interchangeably with cartridge 240
95 Saliva cavity in the casing
100 Structural integrity indicator for the casing 90
105 Casing 90 or cartridge 240 holding a plurality of saliva test strips 120
110 Frustoconical channel for directing saliva 125 of the casing 90
115 Saliva absorbent element of the casing 90 for directing saliva 125 to the saliva test strip 120
120 Saliva test strip
121 Saliva test strip 120 identification code
125 Saliva
130 Immersion of saliva test strip in saliva 125
135 Engagement of saliva test strip 120 to the test element casing 90
140 Saliva test strip 120 disposed within the test element casing 90
145 Enclosure
150 Receiving port of the enclosure 145
155 Color sensing circuitry of the enclosure 145
160 Communication circuitry of the enclosure 145
165 User interface application on the electronic device 60 for use with the enclosure 145
170 User
175 Flow of saliva 125 on the saliva test strip 120 into the test element casing 90 or cartridge 240
180 Camera of the electronic device 60 or camera 220 verifying a user 170 identity usually via facial recognition
185 Camera of the electronic device 60 verifying saliva test strip 120 via an identification code 121
190 Camera of the electronic device 60 getting change of state 55 for saliva test strip 120 test result
195 Continuous dynamic communication of the user 170 identity preferably through facial recognition, also the saliva test strip 120 identification code 121, and saliva strip 120 test results 55 to the internet portal for review by the third party by the electronic device 60 via the internet connection 65
200 A self-contained testing apparatus
205 Body of the self-contained testing apparatus 200
210 Port of the body 205
211 Power switch of the self-contained testing apparatus 200 wherein the power switch 211 is disposed on the body 205
215 Perceptible output of the body 205
220 Camera of the body 205
225 Control circuitry of the body 205 that uses the body camera 220 for confirming user 170 identity preferably through facial recognition, also the control circuitry image/color sensor confirming the saliva test strip 120 identification code 121, and the change of state 55 of the saliva test strip 120 for saliva strip test results 55, all communicated to the perceptible output 215 of the body 205
230 Internet communication circuitry of the body 205 that communicates the control circuitry 225 perceptible output 215 to the internet portal 195 for review by a third party
235 Saliva testing method
240 Cartridge for securing the saliva test strip 120 that can be used interchangeably with casing 90
245 Saliva cavity in the cartridge 240
250 Structural integrity indicator for the cartridge 240
255 Cartridge 240 holding a plurality of saliva strips 120
256 Saliva 125 cavity for cartridge 255
260 Frustoconical channel for directing saliva 125 of the cartridge 240
265 Saliva absorbent element of the saliva test strip 120
270 User interface display application on the electronic device 60 for the complete saliva testing method 235
275 Test stand

### DETAILED DESCRIPTION

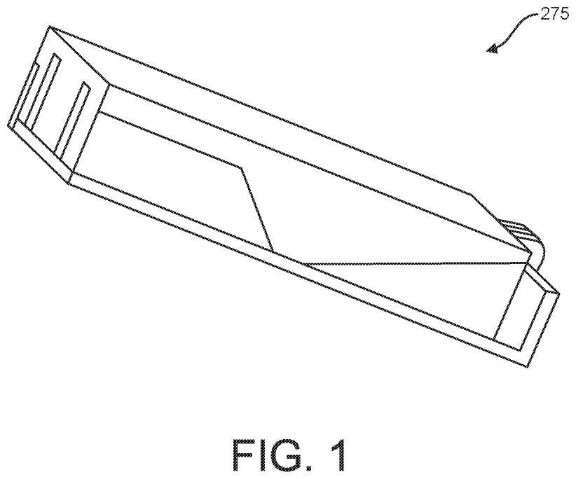

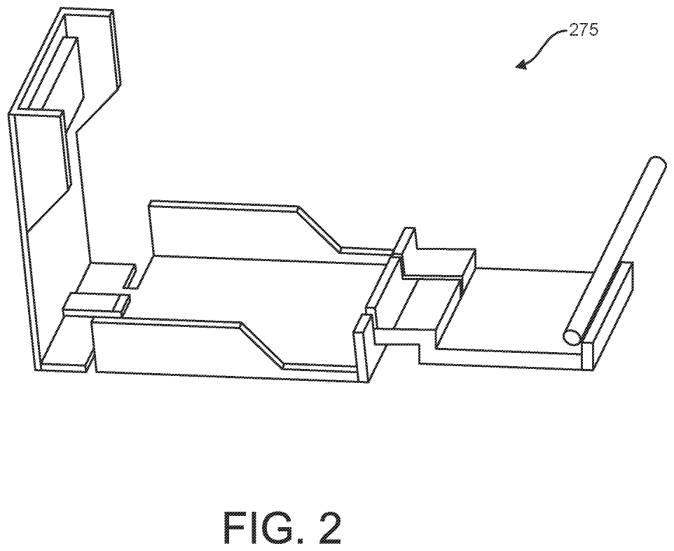

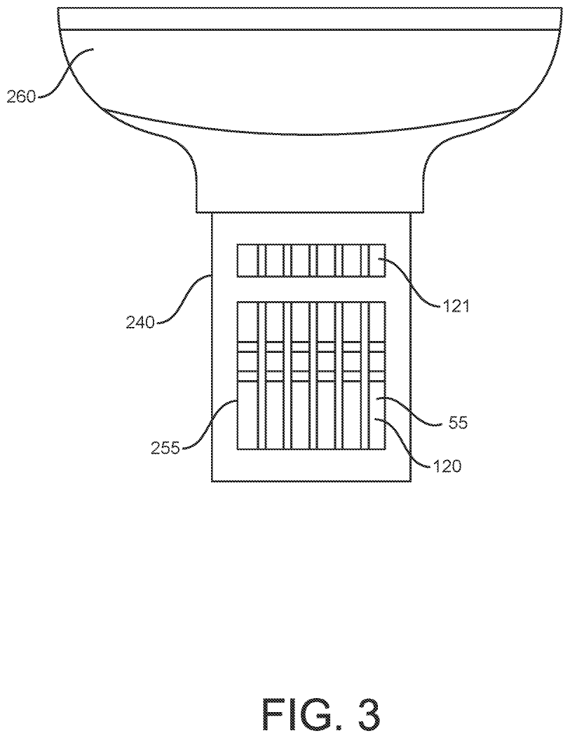

With initial reference to FIG. 1 shown is an upper perspective view of the test stand 275 that is in the folded state, next, FIG. 2 shows an upper perspective view of the test stand 275 that is in the opened state. Further, FIG. 3 shows a side elevation view of the frustoconical channel 260 for directing saliva into the cartridge 240, also shown is the saliva test strip 120 disposed within the cartridge 240, further shown is the saliva test strip identification code 121 and the test strip 120 indicating the present of a selected substance 55 via color coding.

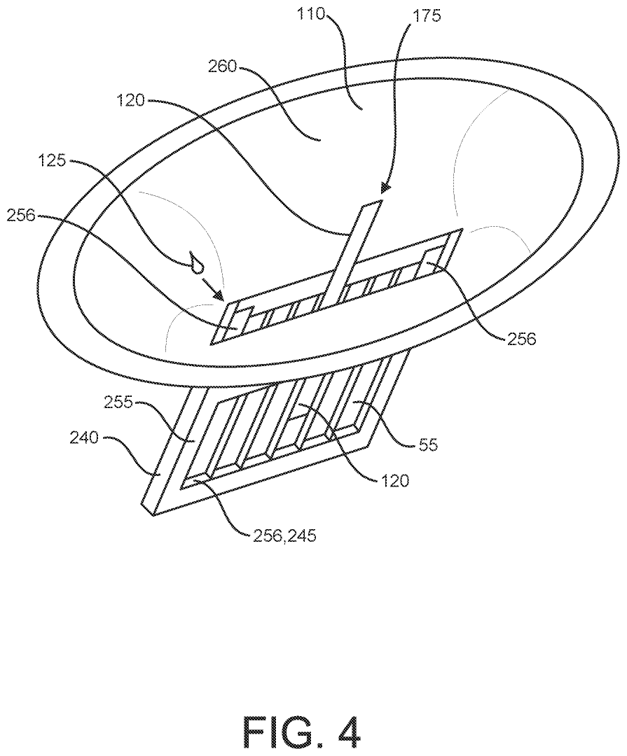

Continuing, FIG. 4 shows an upper perspective view of the frustoconical channel 260 for directing saliva 125 into the cartridge 240, also shown is one of the saliva test strips 120 raised for pictorial clarity, with the saliva test strips 120 all disposed within the cartridge 240, further shown is the saliva test strip 120 identification code 121, with the lower portion of the test strip 120 displaying the color coded test results 55 from the saliva 125 reacting 55 with the test strip 120.

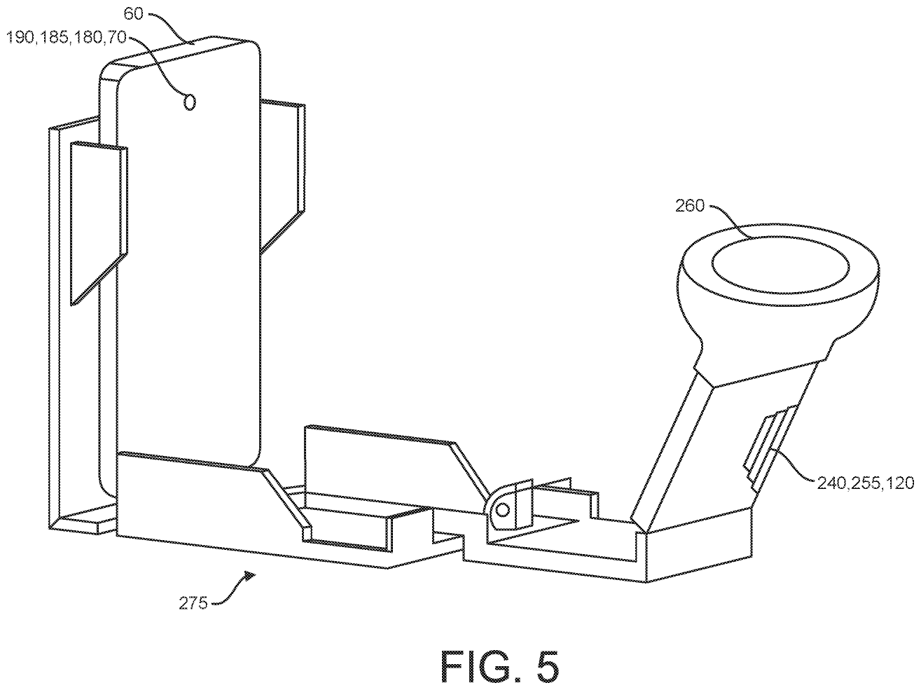

Further, FIG. 5 shows an upper perspective view of the test stand 275 in the opened state wherein an electronic device 60 is disposed in the left side of the stand 275 with the electronic device 60 including a camera 70 facing to the right, further the frustoconical channel 260 for directing saliva 125 into the cartridge 240 is shown disposed in the right side of the test stand 275 wherein the camera 70 faces the cartridge 240 to record the image 55 of the saliva test strip 120 identification code 121 and the saliva test strip 120 color coded test results 55.

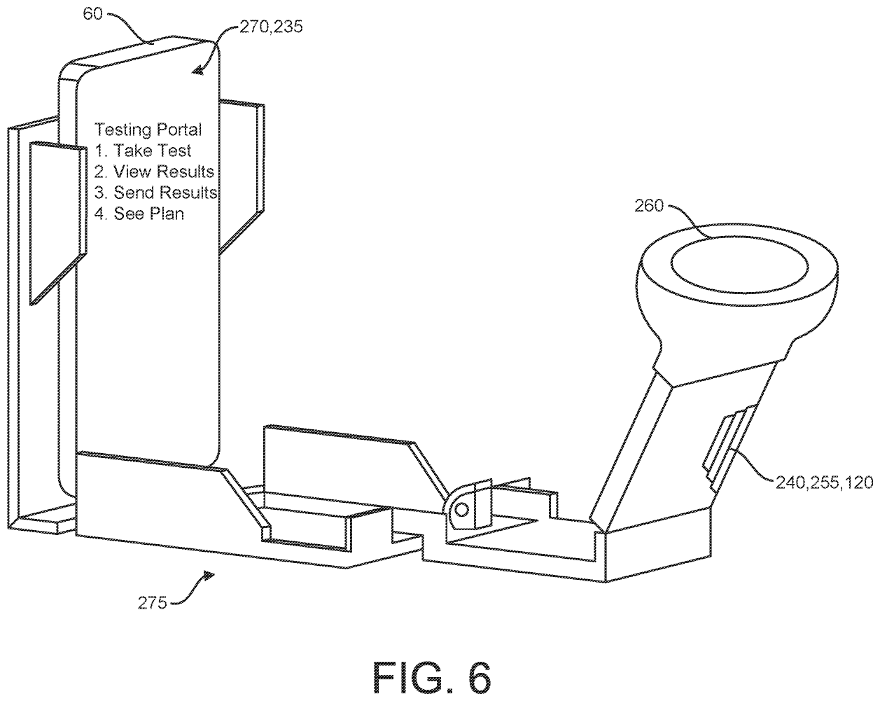

Next, FIG. 6 shows an upper perspective view of the test stand 275 in the opened state wherein the electronic device 60 is disposed in the left side of the stand with the electronic device 60 including the camera 70 facing to the right, further the frustoconical channel 260 for directing saliva 125 into the cartridge 240 is shown disposed in the right side of the test stand 275 wherein the camera 70 faces the cartridge 240 to record the image 55 of the saliva test strip 120 identification code 121 and the saliva test strip 120 color coded test results 55, further the display screen 270 of the electronic device 60 a menu for test instructions 235 as part of the user interface 270.

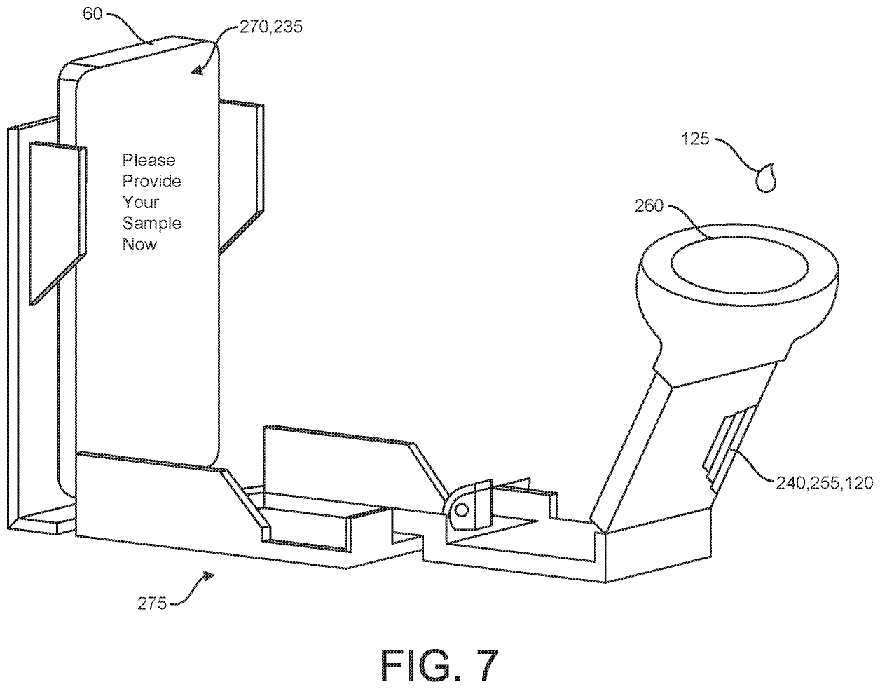

Further, FIG. 7 being a continuation of FIG. 6 shows an upper perspective view of the test stand 275 in the opened state wherein the electronic device 60 is disposed in the left side of the test stand 275 with the electronic device 60 including the camera 70 facing to the right, further the frustoconical channel 260 for directing saliva 125 into the cartridge 240 is shown disposed in the right side of the test stand 275 wherein the camera 70 faces the cartridge 240 to record the image 55 of the saliva test strip 120 identification code 121 and the saliva test strip 120 color coded test results 55, further the display screen 270 of the electronic device 60 a menu for test instructions 235 as part of the user interface 270 with the actual saliva test sample 125 shown going into the frustoconical channel 260.

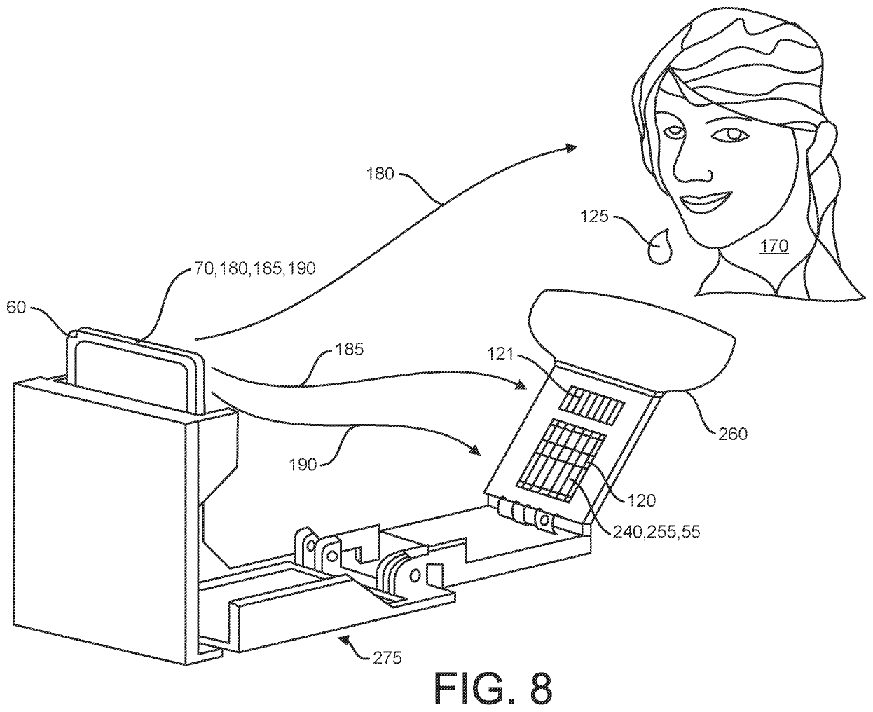

Moving onward, FIG. 8 being a continuation of FIG. 6 shows a reverse upper perspective view of the test stand 275 in the opened state wherein the electronic device 60 is disposed in the left side of the stand with the electronic device 60 including the camera 70 facing to the right, further the frustoconical channel 260 for directing saliva 125 into the cartridge 240 is shown disposed in the right side of the test stand 275 wherein the camera 70 faces the cartridge 240 as shown to record the image 55 of the saliva test strip 120 identification code 121 and the saliva test strip 120 color coded test results 55, with the actual saliva test sample shown going into the frustoconical channel 260.

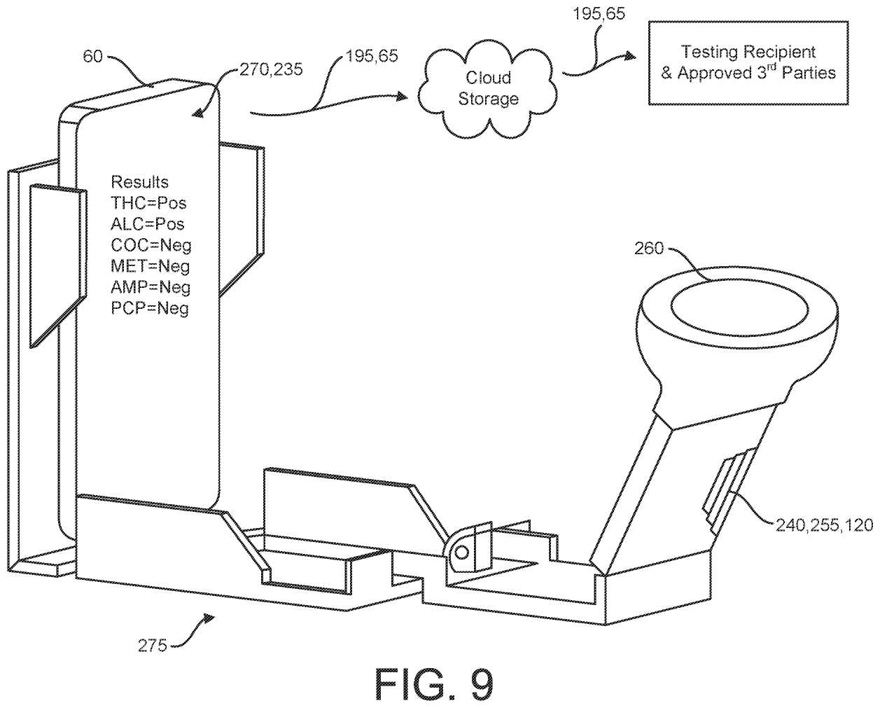

Next, FIG. 9 shows an upper perspective view of the test stand 275 in the opened state wherein the electronic device 60 is disposed in the left side of the stand 275 with the electronic device 60 including the camera 70 facing to the right, further the frustoconical channel 260 for directing saliva 125 into the cartridge 240 is shown disposed in the right side of the test stand 275 wherein the camera 70 faces the cartridge 240 to record the image of the saliva test strip 120 identification code 121 (not shown in FIG. 9) and the saliva test strip 120 color coded test results 55 (not shown in FIG. 9), further the display screen 270 of the electronic device 60 a menu for test instructions 235 as part of the user interface 270, also shown in the electronic device 60 utilizing cloud storage 65, 195 for test results and review by third parties 80.

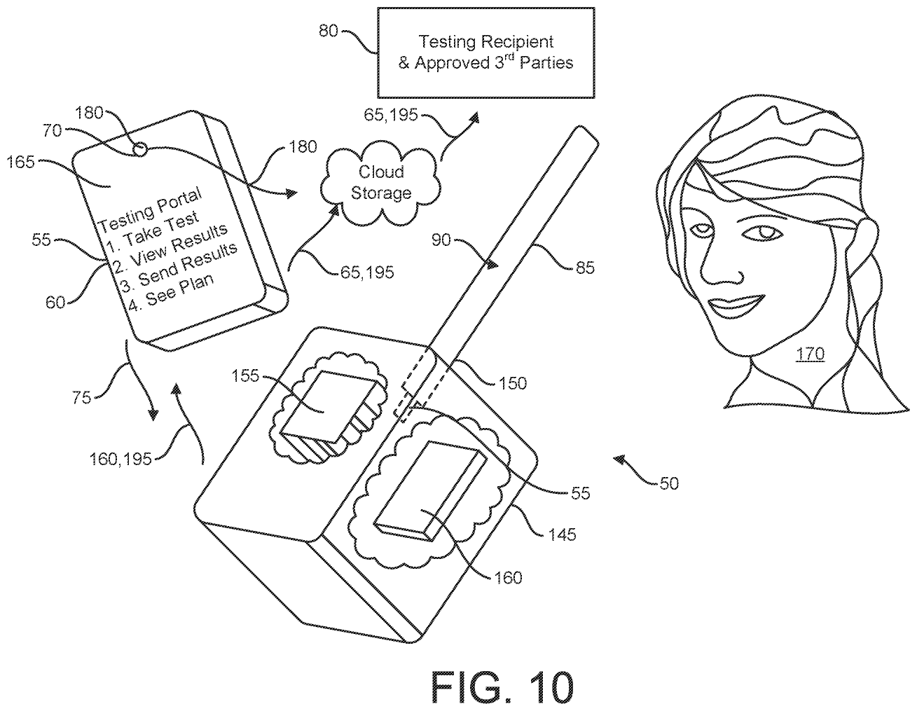

Continuing, FIG. 10 shows an upper perspective view of the ancillary saliva testing apparatus 50 that includes the saliva test element assembly 85, color sensing circuitry 155, communication circuitry 160 all disposed within an enclosure 145, also shown is the electronic device 60 that provides a user interface 270 with the menu 235 shown on the display screen 270, wherein the electronic device 60 communicates 75 with the ancillary saliva testing apparatus 50 and cloud storage 65, 195 for test results and review by third parties 80.

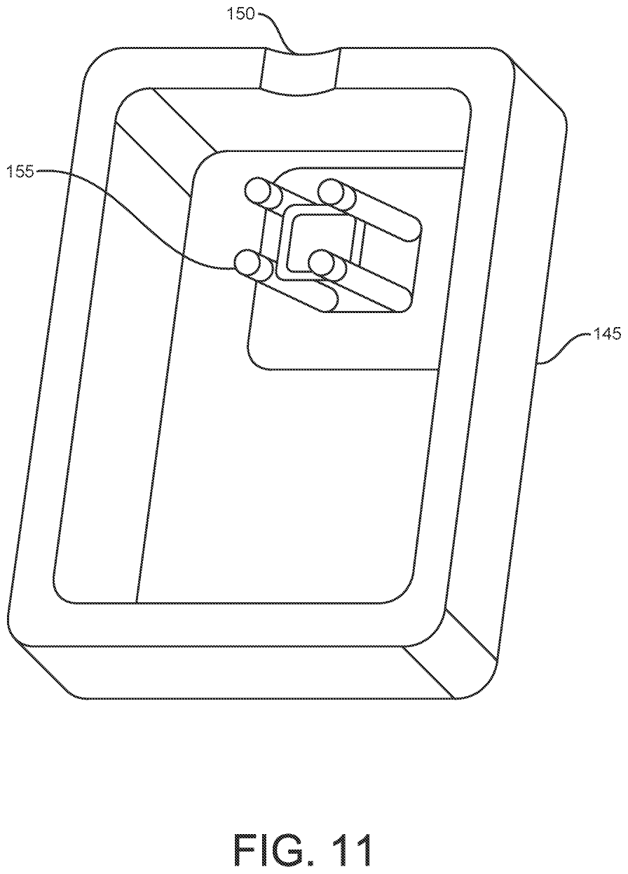

Next, FIG. 11 shows a perspective view of the enclosure 145 interior that includes the color circuitry 155, and a portion of the receiving port 150 of the enclosure 145 for the saliva test element assembly 85.

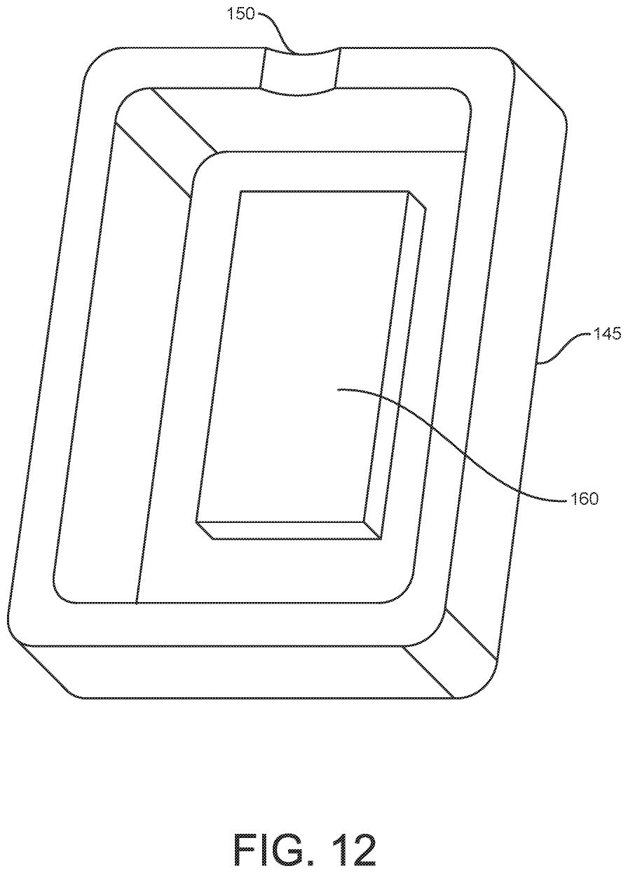

Further, FIG. 12 shows a perspective view of the enclosure 145 interior that includes the communication circuitry 160, and a portion of the receiving port 150 of the enclosure 145 for the saliva test element assembly 85.

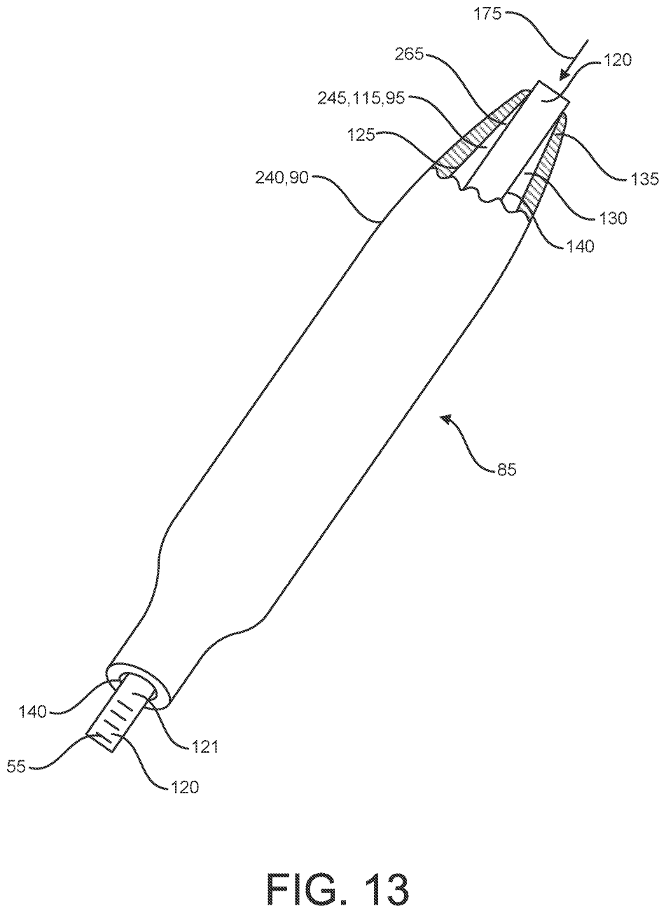

Continuing, FIG. 13 shows a perspective and partial cross sectional view of the saliva test element assembly 85 that includes the element casing 90, the saliva cavity 95, the saliva test strip 120 disposed within the element casing 90, and the saliva test strip identification 121, plus indication of a selected substance 55 on the saliva test strip 120.

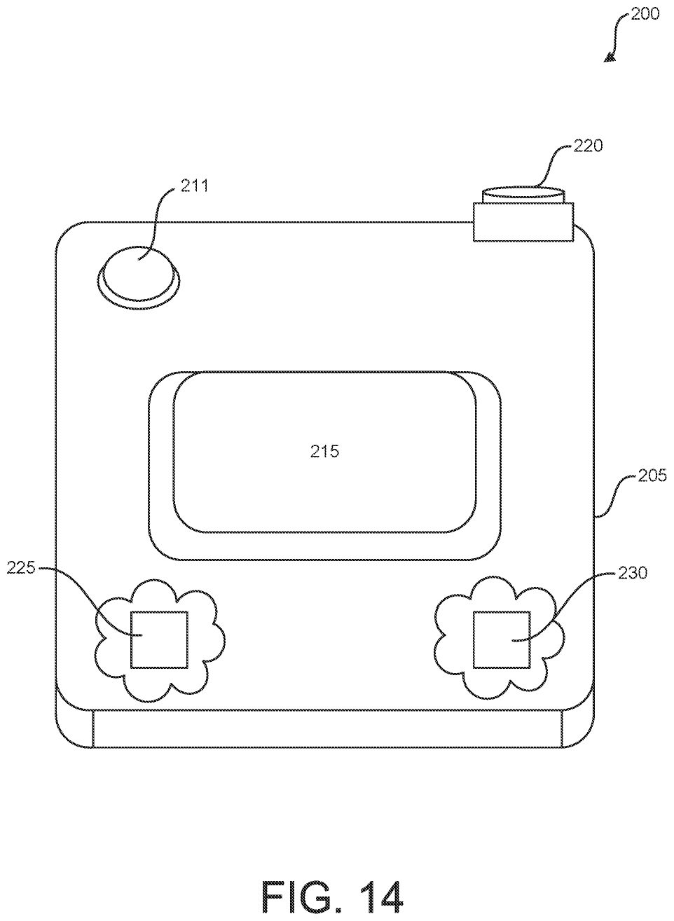

Moving onward, FIG. 14 shows a perspective view of the self-contained testing apparatus 200 that includes the camera body 220, the perceptible output 215, the body 205, the control circuitry 225, and the internet communication circuitry 230, all disposed within the body 205.

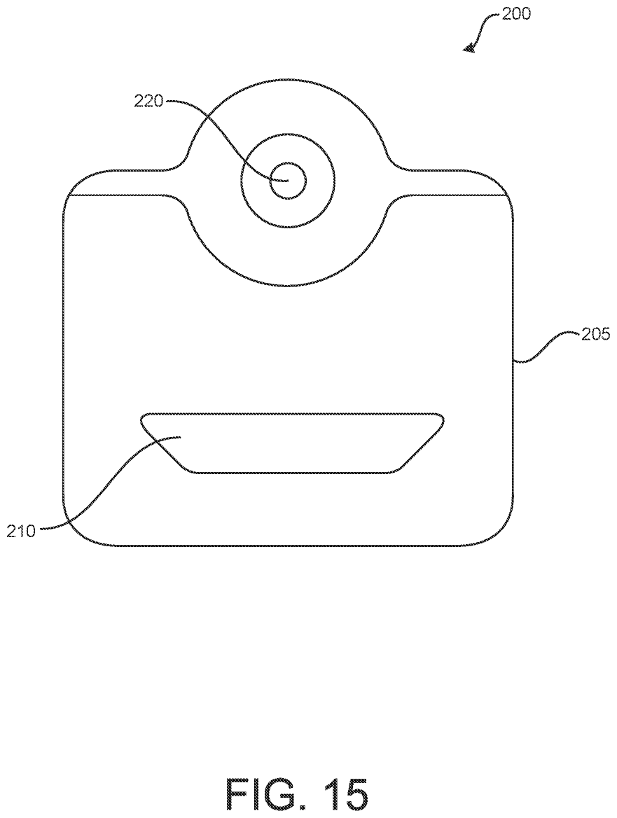

Next, FIG. 15 shows a top view of the self-contained testing apparatus 200 that includes the camera body 220, the port 210 for the saliva test cartridge 240 (not shown), all disposed within the body 205.

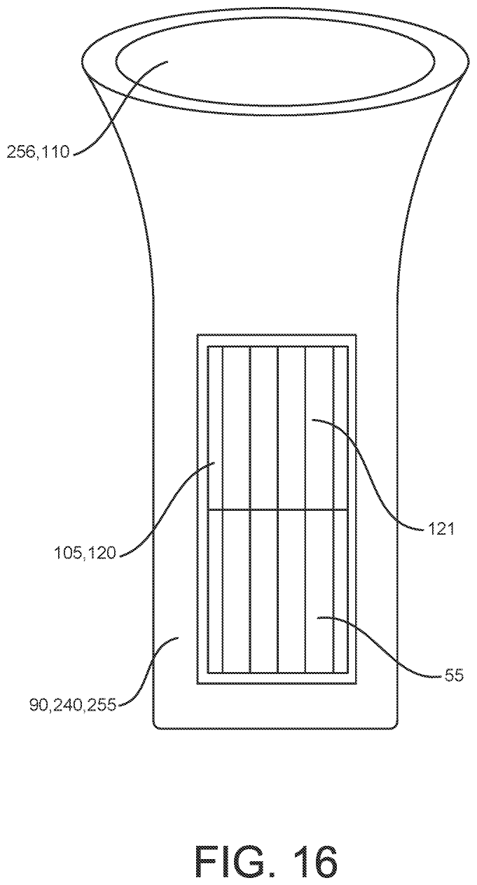

Further, FIG. 16 shows a side elevation view of the frustoconical channel 110 for directing saliva 125 (not shown) into the cartridge 240, also shown are the saliva test strips 120 disposed within the cartridge 240, further the saliva test strip identification codes 121, plus indication of a selected substance 55 on the saliva test strips 120.

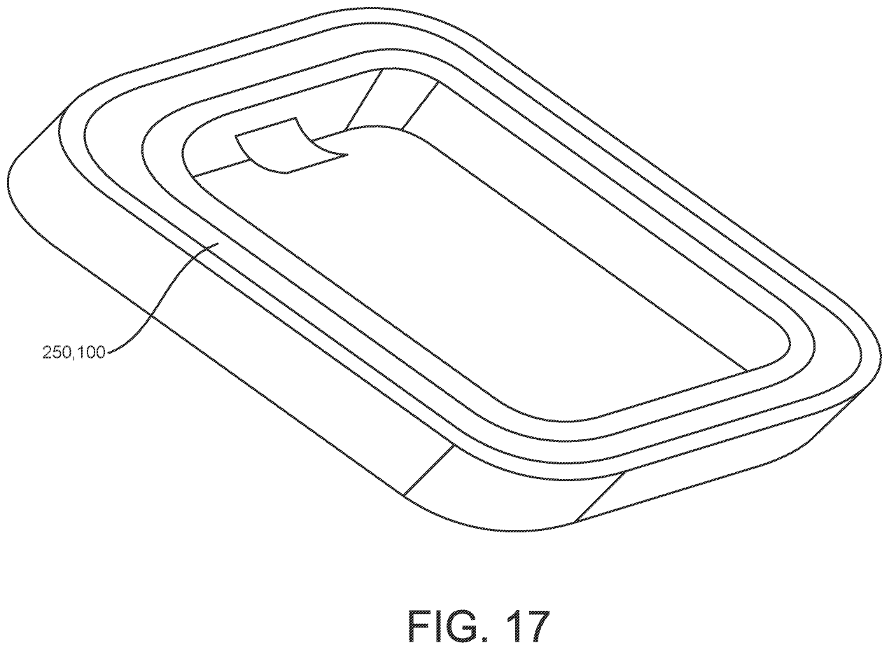

Continuing, FIG. 17 shows a perspective view of the structural integrity indicator 100, 250 respectively for the cartridge 240 or casing 90.

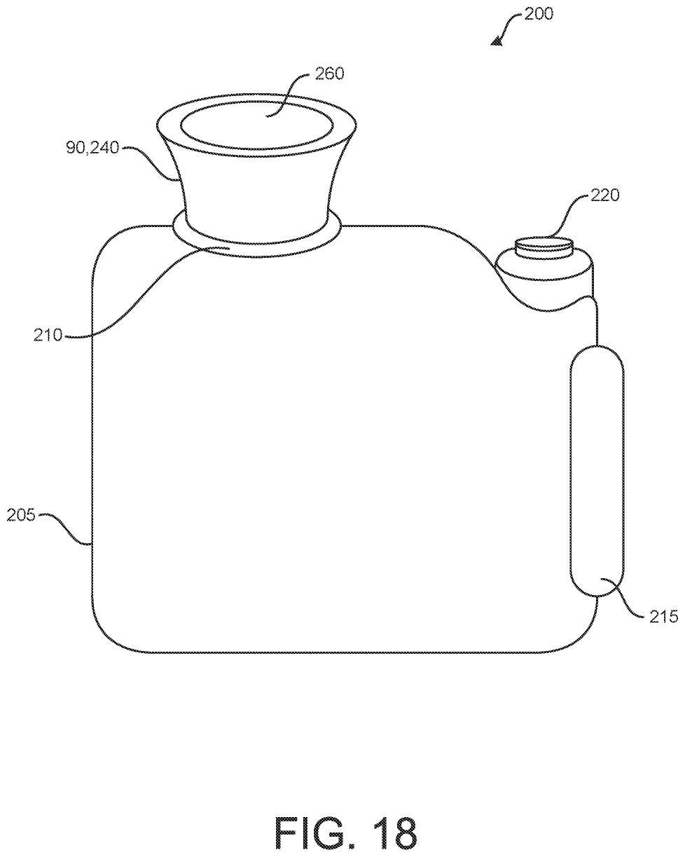

Moving onward, FIG. 18 shows a perspective view of the self-contained testing apparatus 200 that includes the camera body 220 disposed in the body 205, with the frustoconical channel 260 and test strip cartridge 240 inserted into the body 205 port 210.

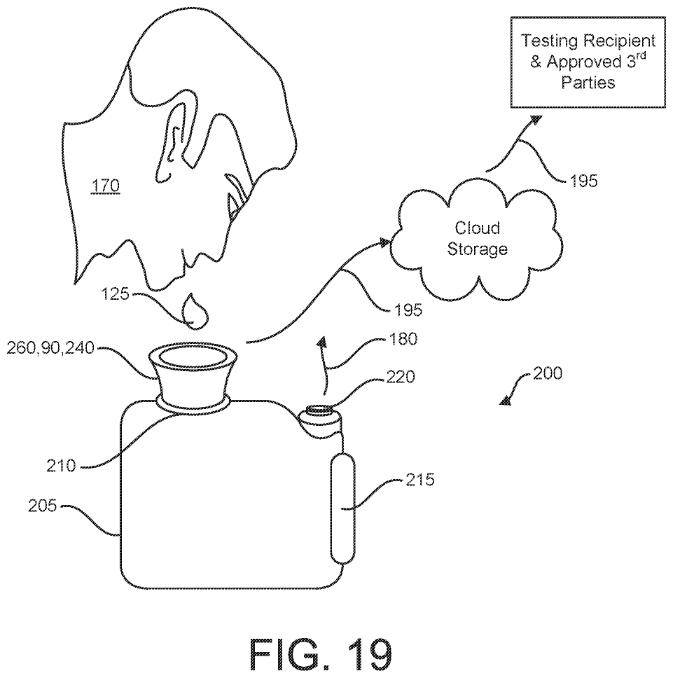

Next, FIG. 19 shows a perspective use view of the self-contained testing apparatus 200 that includes the camera body 220 disposed in the body 205, the perceptible output 215, with the frustoconical channel 260 and test strip cartridge 240 inserted into the body port 210, also shown is the cloud storage 195 for test results and review by third parties 195.

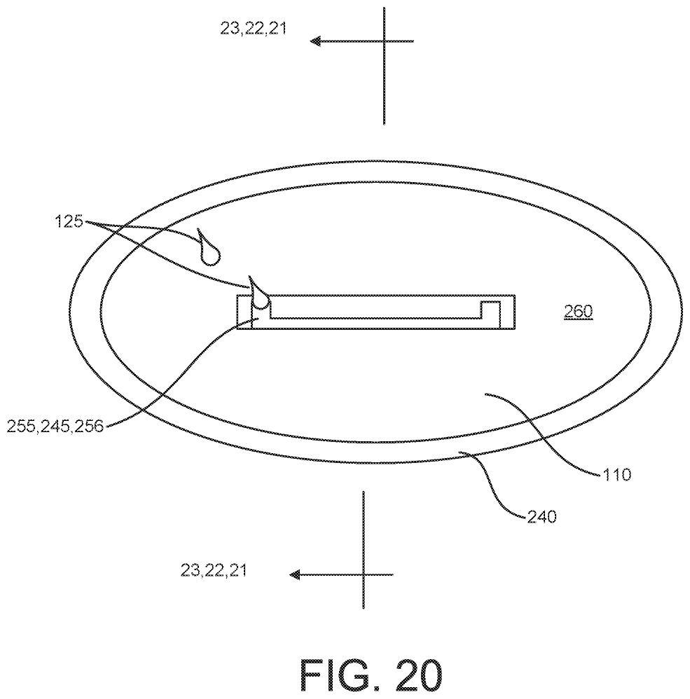

Continuing, FIG. 20 shows a top view of the frustoconical channel 260 for directing saliva 125 into the cartridge 240, also shown is the saliva cavity 256 for the cartridge 255 that is disposed within the cartridge 240, wherein the saliva 125 is directed into the saliva cavity 256.

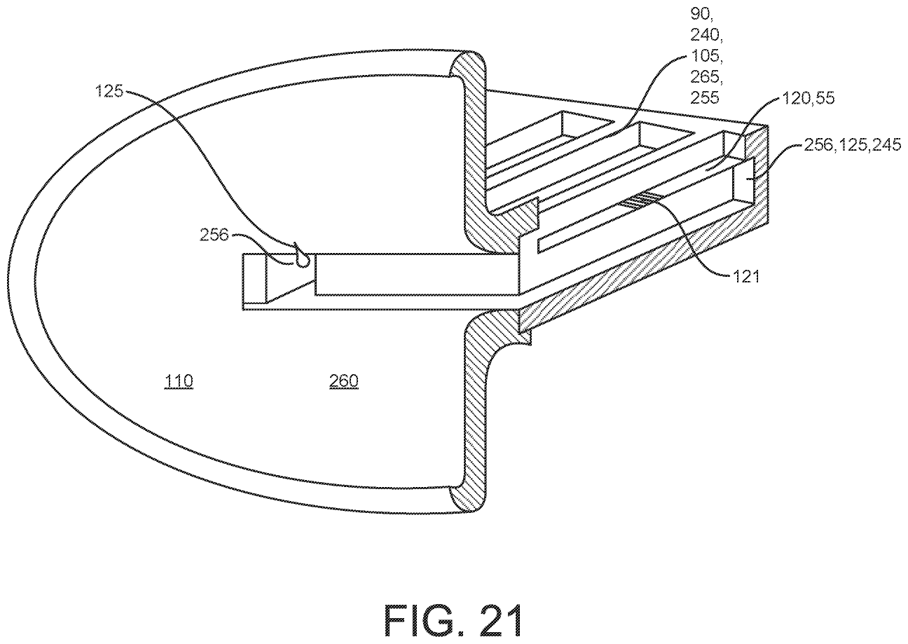

Next, FIG. 21 shows a perspective cross section 21-21 from FIG. 20, wherein FIG. 21 shows the frustoconical channel 260 for directing saliva 125 into the cartridge 240, also shown is the saliva cavity 256 for the cartridge 240 that is disposed within the cartridge 240, also shown is the saliva test strip 120 disposed within the cartridge 240, further the saliva test strip 120 identification code 121, with the lower portion of the test strip 120 displaying the color coded test results 55 for presence of the selected substance from the saliva 125 reacting with the test strip 120.

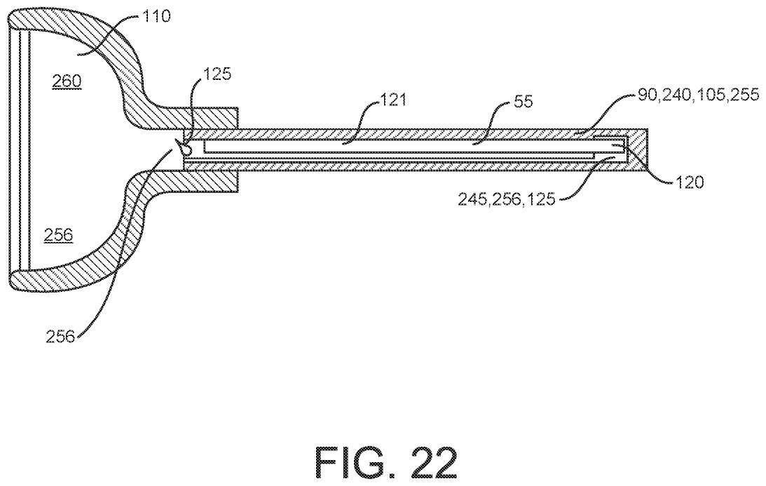

Further, FIG. 22 shows a cross section 22-22 from FIG. 20, wherein FIG. 22 shows the frustoconical channel 260 for directing saliva 125 into the cartridge 240, also shown is the saliva cavity 256 for the cartridge 240, wherein the saliva cavity 256 is disposed within the cartridge 240, also shown is the saliva test strip 120 disposed within the cartridge 240, further the saliva test strip 120 identification code 121, with the lower portion of the test strip 120 displaying the color coded test results 55 for presence of the selected substance from the saliva 125 reacting with the test strip.

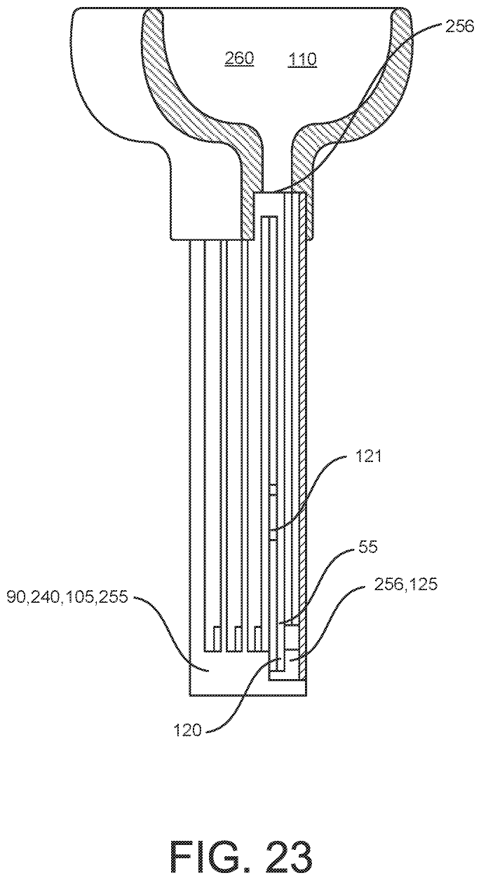

Also, FIG. 23 shows a another perspective cross section 23-23 from FIG. 20, wherein FIG. 23 shows the frustoconical channel 260 for directing saliva 125 into the cartridge 240, also shown is the saliva cavity 256 for the cartridge 240, wherein the saliva cavity 256 is disposed within the cartridge 240, also shown is the capability of the cartridge 240 holding a plurality of saliva test strips 120 that are disposed within the cartridge 240, further the saliva test strip identification code(s) 121, with the lower portion of the test strip 120 displaying the color coded test results 55 for presence of the selected substance(s) from the saliva 125 reacting with the test strip(s) 120.

Broadly, in looking at FIGS. 10 to 13, the present invention is an ancillary saliva testing apparatus 50 for helping to indicate the presence a selected substance 55 that utilizes an internet connected 65 electronic device 60 with a camera 70, wherein the electronic device 60 also having electronic communication 160, 195 to the saliva testing apparatus 50, the saliva testing apparatus 50 includes a saliva test element assembly 85 that includes a test element casing 90 and a consumable saliva test strip 120 that is disposed 140 within the test element casing 90, see in particular FIG. 13.

Further included in the ancillary saliva testing apparatus 50 is the enclosure 145 that includes the receiving port 150 for the test element casing 90, the enclosure 145 further includes color sensing circuitry 155 for the consumable saliva test strip 120, and communication circuitry 160 from the color sensing circuitry to the electronic device 60.

In addition for the ancillary saliva testing apparatus 50 a user interface application 165 is included on the electronic device 60 for the enclosure 145 to operationally facilitate dynamic and continuous documenting 195 a user 170 inserting 175 the saliva test strip 120 that is saliva 125 wetted into the test element casing 90 with the electronic device 60 camera 70, 180 also verifying the user 170 identity preferably by facial recognition. Further for the saliva test strip 120 that includes both the identification code 121 on the saliva test strip 120 and change of state 190 test result color code 55 on the saliva test strip 120, wherein the color sensing circuitry communicates 155 the wetted saliva test strip image to 55, 121 the user interface application 165 through the communication circuitry 160 displaying saliva strip 120 test results from selected saliva test strip 120 test result criteria 55, wherein the test results are further communicated 195 to an internet portal for a third party to have access to the saliva testing results 55.

Optionally for the ancillary saliva testing apparatus 50, the casing 90 can include a saliva cavity 95 for help to make consistent a volume of saliva 125 absorbed by the saliva test strip 120, see in particular FIG. 13.

Another option for the ancillary saliva testing apparatus 50, the casing 90 can have a structural integrity indicator 100 that provides a perceptible output indicating casing 90 opening through a color change, sticker separation, an open electrical circuit, or equivalent, see in particular FIG. 17.

A further option for the ancillary saliva testing apparatus 50, is wherein the casing 90 is sized and configured to hold a plurality 105 of saliva test strips 120 now being termed a cartridge 240, see FIGS. 3 to 9, 16, and 21 to 23.

A continuing option for the ancillary saliva testing apparatus 50, wherein the casing 90 now termed a cartridge 240 can further comprise a frustoconical channel 110 on one end to direct user saliva 125 flow to the saliva test strip 120, see FIGS. 4 and 20 to 22.

Alternatively for the ancillary saliva testing apparatus 50, wherein the casing 90 can further comprise a saliva absorbent element 115 on one end to direct user 170 saliva 125 flow to the saliva test strip 120 from placing the saliva absorbent element 115 in a user's 170 mouth, see FIG. 13.

In looking at FIGS. 3-4 and 14 to 23, a self-contained saliva testing apparatus 200 is disclosed for helping to indicate the presence a selected substance 55, the saliva testing apparatus 200 includes the body 205 that includes the port 210, also the perceptible output 215 disposed on the body 205, further included is the camera 220 disposed within the body 205, the saliva test strip 120, in addition the cartridge 240 that receives the test strip 120, wherein the cartridge 240 includes a saliva cavity 245 for help to make consistent a volume of saliva 125 absorbed by the saliva test strip 120, the cartridge 240 interfacing with the port 210.

Also included in the self-contained saliva testing apparatus 200 is control circuitry 225 for the camera 220 for verifying the user 170 identity preferably by facial recognition, with the control circuitry 225 color sensor confirming the saliva test strip 120 that includes both an identification code 121 on the saliva test strip 120 and change of state test result color code 55 on the saliva test strip 120 that is disposed within the cartridge 240, wherein the cartridge 240 is inserted into the port 210, wherein the control circuitry communicates 225 the user 170 identity, the wetted saliva test strip 120 identification code 121, and test results 55 to the perceptible output 215.

Optionally for the self-contained saliva testing apparatus 200, it can further comprise internet communication circuitry 230 disposed within the body 205 that is operational to facilitate an internet connection 195 for the purpose of communicating the verifying of user 170 identity preferably by facial recognition via camera 220, with the control circuitry 225 color/image sensor confirming the saliva test strip 120 that includes both an identification code 121 on the saliva test strip 120 and change of state test result color code 55 on the saliva test strip 120 results 55 to an internet portal 195 for the third party to have access to the saliva testing results 55.

A further option for the self-contained saliva testing apparatus 200, wherein the casing 90, cartridge 240 can have a structural integrity indicator 100, 250 that provides a perceptible output indicating casing 90, cartridge 240 opening through a color change, sticker separation, open electrical circuit indication, or equivalent, see in particular FIG. 17.

Another option for the self-contained saliva testing apparatus 200, wherein the casing 90, cartridge 240 is sized and configured to hold a plurality 105 of saliva test strips 120, now being termed cartridge 255, see FIGS. 3 to 9, 16, and 21 to 23.

Alternatively, for the self-contained saliva testing apparatus 200, it can further comprise a frustoconical channel 110 on one end to direct user saliva 125 flow to the saliva test strip 120, see FIGS. 4 and 20 to 22.

Further, alternatively for the self-contained saliva testing apparatus 200, wherein the casing 90, cartridge 240, can further comprise a saliva absorbent element 115, 265 on one end to direct user 170 saliva 125 flow to the saliva test strip 120 that can be from placing the saliva absorbent element 115 in a user's 170 mouth, see FIGS. 13 and 21.

Looking at FIGS. 1 to 9, a saliva testing method 235 for helping to indicate the presence a selected substance 55 that utilizes an internet connected 195 electronic device 60 with a camera 70 is disclosed, wherein the saliva testing method comprises the steps of firstly a step of providing the saliva test strip 120 disposed within the casing 90/cartridge 240, wherein the casing 90/cartridge 240 includes the saliva cavity 95, 245 for help to make consistent a volume of saliva 125 absorbed by the saliva test strip 120.

A second step of providing an application 270 operating on the electronic device 60 to complete the saliva 125 testing method that includes the steps of initially having dynamic continuous facial recognition 180 of the user 170 utilizing the camera 70 and subsequently having a step of dynamic continuous image processing 185 of the saliva test strip 120 that includes both the identification code 121 on the saliva test strip 120 and change of state 55 test result color code on the saliva test strip 120. Further subsequently a step of communicating the dynamic continuous facial recognition 180 and the dynamic continuous image processing 185 to an internet portal 65, 195 for the third party to have access to the saliva testing method 235.

Alternatively for the saliva testing method 235, it can further comprise a step of providing a test stand 275 that is sized and configured to hold the electronic device 60 and the cartridge 240 in a positional alignment to operationally facilitate the camera 70 to more accurately record the facial recognition 180 and the saliva test strip 120 identification code 121 on the saliva test strip 120 and change of state test result color code 55 on the saliva test strip 120.

Further alternatively for the saliva testing method 235, the cartridge 240, 255 can be sized and configured to hold a plurality of saliva test strips 120. Also alternatively for the saliva testing method 235, the cartridge 240, 255 can also further comprise a frustoconical channel 260 on one end to direct user 170 saliva 125 flow to the saliva test strip 120, see in particular FIGS. 4, 20, and 21.

Continuing alternatively for the saliva testing method 235, wherein the cartridge 240, 255 can further comprise a saliva absorbent element 265 on one end to direct user saliva 125 flow to the saliva test strip 120.

Moving further alternatively for the saliva testing method 235, wherein the cartridge 240, 255 can have a structural integrity indicator 250 that provides a perceptible output indicating cartridge 240 opening through a color change, sticker separation, open electrical circuit, or equivalent, see in particular FIG. 17.

In the summary above and in this detailed description, and the claims below, and in the accompanying drawings, reference may be made to particular features of the invention. It may be to be understood that the disclosure of the invention in this specification includes all possible combinations of such particular features. For example, where a particular feature may be disclosed in the context of a particular aspect or embodiment of the invention, or a particular claim, that feature can also be used, to the extent possible, in combination with and/or in the context of other particular aspects and embodiments of the invention, and in the invention generally.

Where reference may be made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

“Exemplary” may be used herein to mean “serving as an example, instance, or illustration.” Any aspect described in this document as “exemplary” may not be necessarily to be construed as preferred or advantageous over other aspects.

Throughout the drawings, like reference characters are used to designate like elements. As used herein, the term “coupled” or “coupling” may indicate a connection. The connection may be a direct or an indirect connection between one or more items. Further, the term “set” as used herein may denote one or more of any item, so a “set of items” may indicate the presence of only one item, or may indicate more items. Thus, the term “set” may be equivalent to “one or more” as used herein.

Alternate embodiments and variations are also contemplated. In the description thus far, the non-limiting embodiment described a device with a color/image sensor. Alternate non-limiting embodiments may replace the color/image sensor and include such color detectors as a photo gate, photoresistor, or any other color detection hardware. Alternate embodiments of the micro processing chip are also contemplated and may include and not be limited ESP32, Devkit V1, Arduino, or any other wireless communication enabled micro processing chip. The device currently is used for detecting cotinine, which is a nicotine metabolite. However, the device has the ability to test for any drug detectable through metabolites in saliva. Some of these may include and are not limited to Cannabis (THC), cocaine, methamphetamine, alcohol, benzodiazepines, barbiturates, MDMA, opiates, opioids, PCP, buprenorphine, and methadone. The device may also be used for any test that could utilize the digitization of saliva test strip results, such as and not limited to, ketones. The above described embodiment connects device to a smartphone with bluetooth. Alternate means to connect device to smartphone may include and not be limited to WiFi and a cable, such as micro USB, USBC, Lightning, or any suitable equivalent.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but may be not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description may be regarded as illustrative instead of restrictive of the present invention.

### CONCLUSION

Accordingly, the present invention of a saliva test apparatus and method has been described with some degree of particularity directed to the embodiments of the present invention. It should be appreciated, though; that the present invention is defined by the following claim construed in light of the prior art so modifications or changes may be made to the exemplary embodiments of the present invention without departing from the inventive concepts contained therein.

1. An ancillary saliva testing apparatus for helping to indicate the presence of a selected substance that utilizes an internet connected electronic device with a camera, wherein the electronic device also having electronic communication to said saliva testing apparatus, said saliva testing apparatus comprising:
(a) a saliva test element assembly that includes a test element casing and a consumable saliva test strip disposed within said test element casing;
(b) an enclosure that includes a receiving port for said test element casing, said enclosure further includes color sensing circuitry for said consumable saliva test strip, and communication circuitry from said color sensing circuitry to the electronic device; and
(c) a user interface application on the electronic device for the enclosure to operationally facilitate dynamic and continuous documenting a user including the user's identification, inserting said test element casing into said enclosure with the electronic device camera verifying the user identity through facial recognition, also for said saliva test strip that includes both an identification code on said saliva test strip and change of state test result color code on said saliva test strip after immersion of said test element casing into said enclosure, wherein said color sensing circuitry communicates said saliva test strip identification code and said wetted saliva test strip test results image to said user interface application through said communication circuitry displaying saliva strip test results from selected saliva test strip test result criteria, wherein said test results are further communicated to an internet portal for a third party to have access to said saliva testing results.

2. An ancillary saliva testing apparatus according to claim 1 wherein said casing includes a saliva cavity for help to make consistent a volume of saliva absorbed by the said saliva test strip.

3. An ancillary saliva testing apparatus according to claim 1 wherein said casing has a structural integrity indicator that provides a perceptible output indicating casing opening.

4. An ancillary saliva testing apparatus according to claim 1 wherein said casing is sized and configured to hold a plurality of saliva test strips.

5. An ancillary saliva testing apparatus according to claim 4 wherein said casing further comprises a frustoconical channel on one end to direct user saliva flow to said saliva test strip.

6. An ancillary saliva testing apparatus according to claim 1 wherein said casing further comprises a saliva absorbent element on one end to direct user saliva flow to said saliva test strip from placing said saliva absorbent element in a user's mouth.

7. A self-contained saliva testing apparatus for helping to indicate the presence of a selected substance, said saliva testing apparatus comprising:
(a) a body that includes a port;
(b) a perceptible output disposed on said body;
(c) a camera disposed within said body;
(d) a saliva test strip;
(e) a cartridge that secures said test strip, wherein said cartridge includes a saliva cavity for help to make consistent a volume of saliva absorbed by the said saliva test strip, said cartridge interfacing with said port; and
(f) control circuitry for said camera for verifying a user identity through facial recognition, said control circuitry includes color/image sensor for confirming said saliva test strip that includes both an identification code on said saliva test strip and change of state test result color code on said saliva test strip, wherein said cartridge is inserted into said port, wherein said control circuitry communicates said user identity, said wetted saliva test strip identification code, and test results to said perceptible output.

8. A self-contained saliva testing apparatus according to claim 7 further comprising internet communication circuitry disposed within said body that is operational to facilitate an internet connection for the purpose of communicating said verifying of user identity via facial recognition, confirming said saliva test strip that includes both an identification code on said saliva test strip and change of state test result color code on said saliva test strip saliva test strip results to an internet portal for a third party to have access to said saliva testing results.

9. A self-contained saliva testing apparatus according to claim 7 wherein said cartridge has a structural integrity indicator that provides a perceptible output indicating cartridge opening.

10. A self-contained saliva testing apparatus according to claim 7 wherein said cartridge is sized and configured to hold a plurality of saliva test strips.

11. A self-contained saliva testing apparatus according to claim 10 wherein said cartridge further comprises a frustoconical channel on one end to direct user saliva flow to said saliva test strip.

12. A self-contained saliva testing apparatus according to claim 7 wherein said cartridge further comprises a saliva absorbent element on one end to direct user saliva flow to said saliva test strip.

13. A saliva testing method for helping to indicate the presence of a selected substance that utilizes an internet connected electronic device with a camera, said saliva testing method comprising:
(a) providing a saliva test strip that is secured within a cartridge, wherein said cartridge includes a saliva cavity for help to make consistent a volume of saliva absorbed by said saliva test strip;
(b) providing an application operating on the electronic device to complete said saliva testing method that includes:
(b)(i) dynamic continuous facial recognition of a user utilizing the camera;
(b)(ii) dynamic continuous image processing of said saliva test strip that includes both an identification code on said saliva test strip and change of state test result color code on said saliva test strip while said saliva test strip is secured in said cartridge utilizing the camera; and
(b)(iii) communicating said dynamic continuous facial recognition and said dynamic continuous image processing to an internet portal for a third party to have access to said saliva testing method and results.

14. A saliva testing method according to claim 13 further comprising a step of providing a test stand that is sized and configured to hold the electronic device and said cartridge in a positional alignment to operationally facilitate the camera to more accurately record the facial recognition and said saliva test strip identification code on said saliva test strip and change of state test result color code on said saliva test strip.

15. A saliva testing method according to claim 13 wherein said cartridge is sized and configured to hold a plurality of saliva test strips.

16. A saliva testing method according to claim 15 wherein said cartridge further comprises a frustoconical channel on one end to direct user saliva flow to said saliva test strip.

17. A saliva testing method according to claim 13 wherein said cartridge further comprises a saliva absorbent element on one end to direct user saliva flow to said saliva test strip.

18. A saliva testing method according to claim 13 wherein said cartridge has a structural integrity indicator that provides a perceptible output indicating cartridge opening.
